(19) United States
(12) Patent Application Publication
(10) Pub. No.: US 2026/0354775 A1
(43) Pub. Date: Nov. 12, 2026

(54) METHOD AND SYSTEM FOR OPERATING A COMPUTED TOMOGRAPHY DEVICE

(71) Applicant: Siemens Healthineers AG, Forchheim (DE)

(72) Inventors: Benjamin SCHWEIKERT, Gutenstetten (DE); Michael HOSEMANN, Erlangen (DE); Niklas REHFELD, Nuernberg (DE); Stefan HARTMANN, Eggolsheim (DE)

(73) Assignee: Siemens Healthineers AG, Forchheim (DE)

(21) Appl. No.: 19/671,446

(22) Filed: May 8, 2026

(30) Foreign Application Priority Data
May 9, 2025 (DE) 10 2025 117 940.9

(51) Int. Cl. A61B 6/03; A61B 6/00

(52) U.S. Cl. CPC A61B 6/032; A61B 6/56

(57) ABSTRACT

One or more example embodiments relates to a method for operating a computed tomography device that has a stationary portion, a rotatable portion that is rotatable relative to the stationary portion, and a communication path including a wireless communication link between the stationary portion and the rotatable portion, wherein the rotatable portion has an X-ray detector configured to acquire raw data and a first processing device configured to determine detector data based on the raw data, wherein the stationary portion has a second processing device configured to process the detector data. During an imaging procedure, the detector data is divided by way of a selection unit of the first processing device into first detector data and second detector data. The second detector data is transferred in compressed form in a second data stream via the communication path from the first processing device to the second processing device.

### CROSS-REFERENCE TO RELATED APPLICATION(S)

The present application claims priority under 35 U.S.C. § 119 to German Patent Application No. 10 2025 117 940.9, filed May 9, 2025, the entire contents of which is incorporated herein by reference.

### FIELD

One or more example embodiments relates to a method and a system for operating a computed tomography device (also referred to as a CT device), a control means for a computed tomography device and a computed tomography device.

### RELATED ART

Computed tomography devices are already well known in the art and conventionally have at least one capture assembly that is rotatable about a patient or other object to be examined and has an X-ray emitter and an X-ray detector. The capture assembly with X-ray emitter and X-ray detector is thus part of a rotatable portion of the computed tomography device, which may for example be guided in a computed tomography device gantry that is part of the stationary portion. To be able to determine CT image data sets, raw data captured with the at least one X-ray detector is conventionally processed locally using a processing means to yield detector data that is transferred to a computing means of the computed tomography device that is part of the stationary portion and often also denoted image processor. The image processor uses the detector data to reconstruct a CT image data set, for example a set of sectional images and/or a three-dimensional image volume.

The X-ray detector of a computed tomography device typically comprises a detector pixel array corresponding to a two-dimensional arrangement of detector pixels, wherein the detector pixels are not necessarily arranged in one plane, but rather may also be arranged on a curved surface, for example part of an inner surface of a spherical surface or cylindrical surface. The detector pixels may also be arranged in pixel groups, such that the detector pixel array contains an array of pixel groups, wherein each pixel group in turn contains a subarray of individual detector pixels. The individual detector pixels of a pixel group may, for example, adjoin one another directly, while spaces may be provided between different pixel groups. Other detector pixel array architectures are possible, however. It is also possible for the detector pixel array to have a plurality of different hardware components that are likewise arranged in the form of an array. Each hardware component then contains a plurality of detector pixels that may be combined within the hardware component to form pixel groups. During capture, all of these detector pixels are repeatedly read out and all this data has to be transferred.

To ensure maximum freedom of movement of the rotatable portion, in order in particular to allow rotational passes over large angular ranges, for example complete revolutions, it has been proposed to provide a wireless communication link on the communication path from the processing means to the computing means.

Technical considerations mean, however, that the maximum transfer rate of the wireless communication link is limited, meaning, for example, that at most 25 to 30 gigabits per second can be transferred. The data rate via a slip ring is, however, also limited. Current X-ray detectors, in particular “counting” X-ray detectors (counting detectors), produce raw data internally at a markedly higher data rate, in particular when a plurality of X-ray detectors are provided. This may be the case, for example, if the computed tomography device is a biplane CT device and/or a dual-source CT device, in which the rotatable portion has two capture assemblies at an angle to one another with respective X-ray detectors. For example, raw data rates of multiple hundred Gbit/s can be achieved with two counting detectors.

In CT devices, and in X-ray devices in general, there is in any event a need for the administered X-rays to be translated into a diagnostically useful X-ray image. This means that, even in the event of system failure, for example a power outage, an error along the communication path and/or other abnormal termination, captured detector data must nonetheless be persistently stored in the computing means and it must be possible to reconstruct a CT image data set. This means that real-time transfer of the detector data must be ensured.

To allow this, in known CT devices the processing means processes the raw data such that a data stream of detector data is obtained that has a transfer rate (data rate) that is less than or equal to the maximum transfer rate. To this end, it is known, for example, to reduce the spatial resolution of the processing means, in the case of counting detectors to select and transfer detector data associated with only a small number of energy threshold values, and/or to use compression methods. The latter methods include, for example, hybrid data coding and differential statistical data coding. To reduce spatial resolution, it is known, for example, always to combine four (2×2) pixels on the X-ray detector. This process may also be known as “fusing”.

This makes it possible to meet the requirements of real-time transfer and to produce a data stream of detector data, the transfer rate of which in the case of direct transfer (real-time transfer) is less than or equal to the maximum transfer rate of the wireless communication link. After each time step, in which the at least one X-ray detector is read out, measured information can be sent in very small packets and stored on the image processor, i.e., the computing means.

Should transient transfer errors occur, it has been proposed to use a FIFO buffer to enable resending. While this may result in relatively short delays, if raw data acquisition is terminated abnormally, the previously measured detector data is already present on the computing means, apart from possibly a very small number of small data packets.

In known CT devices, this leads to severe limitations if highly accurate information is required. For example, it is known that, in return for also being able to make use of the high spatial resolution of the at least one X-ray detector, the number of transferred detector rows is limited in order to maintain the maximum transfer rate. The use of further energy threshold values of the at least one X-ray detector may, for example result in the need for a more marked reduction in spatial resolution.

Reference is made to EP 4 434 466 A1, which provides for separate data dispatch. In this case, one part of the data is transferred in a critical time interval and another part in a non-critical time interval.

### SUMMARY

One or more example embodiments provides a method and a system for operating a computed tomography device, a control means for a computed tomography device, and a computed tomography device which overcome the above-described disadvantages. In particular, it is an object of one or more example embodiments of the invention to also enable more data-intensive imaging procedures in CT devices despite the use of a wireless communication link and the need for a computed tomography image to be available even in the event of abnormal termination or system failure.

### BRIEF DESCRIPTION OF THE DRAWINGS

Example embodiments of the present invention are explained below with reference to the appended figures. In the various figures, identical components are here provided with identical reference signs. The figures are not in general to scale. In the figures:

FIG. 1 shows a method according to an exemplary embodiment of the invention;

FIG. 2 shows an outline of a data flow according to the method of FIG. 1;

FIG. 3 shows a schematic outline of an X-ray means according to an exemplary embodiment of the invention;

FIG. 4 shows the functional structure of the computed tomography device and its components according to an exemplary embodiment of the invention;

FIG. 5 shows a method for compressing data from a computed tomography device according to an exemplary embodiment of the invention;

FIG. 6 shows a method for generating a reconstructed CT image according to an exemplary embodiment of the invention;

FIG. 7 shows a method for compressing data from a computed tomography device according to an exemplary embodiment of the invention;

FIG. 8 shows a method for compression and decompression of data from a computed tomography device according to an exemplary embodiment of the invention; and

FIG. 9 shows a schematic representation of an artificial neural network according to an exemplary embodiment of the invention.

### DETAILED DESCRIPTION

A method according to one or more example embodiments serves to operate a computed tomography device having a stationary portion and a portion that is rotatable relative to the stationary portion, wherein the rotatable portion has at least one X-ray detector, wherein the stationary portion has a computing means (also referred to as a second processing device) configured to process detector data, wherein the at least one X-ray detector has an assigned processing means (also referred to as a first processing device) for determining, on the basis of raw data from the at least one X-ray detector, detector data to be transferred via a communication path to the computing means and the communication path has a wireless communication link with a maximum transfer rate, wherein, in an imaging procedure,
a selection unit of the processing means is used to divide the detector data into a first data stream, the first detector data of which is sufficient, after conclusion of raw data acquisition using the at least one X-ray detector, for reconstruction of an evaluatable CT image data set (computed tomography data set), and which has a data transfer rate corresponding at most to the maximum transfer rate, and into a second data stream comprising the remaining detector data as second detector data,
the first data stream is transmitted directly as a real-time transfer via the communication path to the computing means and
the second detector data is temporarily stored in a temporary storage means and, after conclusion of raw data acquisition using the at least one X-ray detector and transfer of the first data stream, is transferred via the communication path to the computing means.

Such division of the detector data is known from EP 4 434 466 A1. A feature of one or more example embodiments of the invention, however, is that, in contrast thereto, precompressed data is generated prior to transfer by compression of the second detector data using a lossy compression method, and fully compressed data is provided from the precompressed data using a lossless compression method.

In the method, all the detector data from a capture is thus sent in the form of two data streams. The processing means and the communication path are important for this purpose. The processing means serves to divide the detector data using a selection unit (also referred to as a selector), the data then being sent in the form of first detector data and second detector data via the communication path (for example, a data transfer network). The communication path here comprises a wireless communication link such as for example a radio network or a slip ring.

Using the selection unit, the detector data is divided into first detector data and second detector data. The first detector data is here selected such that a usable CT image can be reconstructed from it. The second detector data comprises all the other data from the capture. The first detector data is transmitted in the form of a real-time transfer as a first data stream and could thus also be designated an “online data set”, since it is basically dispatched during capture (or at least directly thereafter). The second detector data is initially temporarily stored and could be designated an “offline data set”, since it is not transferred until capture is concluded. As a rule, the offline data set is much larger than the online data set, meaning that compression thereof here offers a major advantage for the transfer. It should be noted, however, that the first data can also be initially compressed and then dispatched. In particular, compression of the first data is preferred, as described below for the second data.

It should be noted in this respect that, in terms of data rate, it is the communication path that constitutes the system bottleneck. Data processing and storage may be carried out at much higher processing rates than those at which data can be sent.

The detector data of the second data stream is preferably also stored in the temporary storage means in real time, such that the raw data stream arising in the at least one X-ray detector can be processed in real time by the processing means and the resultant detector data can be divided into the data streams using the selection unit. This therefore results in continuous real-time data throughput. In this case, the necessary data transfer rate for unbuffered transfer (real-time transfer) of the first and second data streams would be jointly greater than the maximum transfer rate. It is in particular even conceivable for the data transfer rate for unbuffered transfer (real-time transfer) of the second data stream alone to be greater than the maximum transfer rate.

The concept underlying one or more example embodiments of the invention is that of presorting the detector data in such a way that important first detector data that is sufficient for determining a CT image data set is initially transferred directly and unbuffered to the computing means, i.e., the image processor, and stored therein, while second detector data is assigned a lower importance in terms of the requirement for sufficient detector data to be available for reconstruction of a CT image data set and is initially temporarily stored so as to be transferred as a follow-on. This means that transfer of the detector data in the first data stream and transfer of the second detector data take place in succession. In this way, it is possible to ensure that complete measurement takes place at all times and portions that are sufficient for determining a CT image data set are sent immediately to the computing means and stored therein. The fact that the first detector data is sufficient for reconstruction of an evaluatable CT image data set after conclusion of raw data acquisition using the at least one X-ray detector may be taken to mean that it meets a specified minimum data criterion. Further information is sent to the computing means only after the end of raw data acquisition (and transfer of the first detector data). This follow-on transfer increases the volume of data and thus also the information content of the available detector data. As a result of the performed selection and the time delay, more detector data may thus be transferred, wherein a complete CT image data set is nonetheless available for diagnosis directly at the end of raw data acquisition (end of scan). Further information, such as for example higher spatial resolution or further spectral information, is available after a delay and may accordingly be put to subsequent use.

To carry out this presorting of detector data, it is proposed to make changes directly to the processing means, a selection unit being proposed to create two different data streams. The first data stream is provided for direct transfer to the image processor, i.e., the computing means, while the second data stream is forwarded, for example by way of an appropriate temporary storage unit, for temporary storage in a temporary storage means, ready for delayed transfer after the end of raw data acquisition. As already described, these two data streams are in this case so to speak synchronously filled.

While hitherto the data rate was limited by the maximum transfer rate for direct transfer and higher data rates were consequently impossible, the solution presented here may enable data rates or volumes of data which could not previously be achieved using current data transfer technology. To achieve this, it is merely necessary to provide a suitably large temporary storage means in the rotatable portion, optionally as part of the processing means.

In this respect, it should additionally be generally noted that the X-ray detector may in particular be a “counting detector”, i.e., an X-ray detector configured to count individual photons. A counting detector may in particular be configured not only to detect individual quanta but also additionally to quantify the photon's energy, so enabling spectral imaging, i.e., imaging with energy resolution. To this end, it is known, for example, to use different energy threshold values in the X-ray detector. Generally speaking, the CT device may be a dual-source CT device and/or a biplane CT device, in which the rotating portion consequently includes two capture assemblies each with an X-ray emitter and an X-ray detector. These may, for example, be arranged rotated by 90° relative to one another. In this case, two X-ray detectors are thus present, the raw data of which can be processed for transfer to the computing means in a common processing means or indeed in separate processing means.

Generally speaking, for the purposes of the present invention, there are various possible options as to how first detector data for the first data stream can specifically be selected, for example also in terms of previously undertaken processing by the processing means. In particular, spatial, temporal, and/or spectral reduction may take place compared with the complete detector data. It goes without saying that a combination of these options may also be used. In the case, in particular, of a combination of such reduction techniques, it is particularly advantageously possible for division to be performed in such a way as to make the most extensive possible use of the maximum transfer rate. This means it may be expedient, as early as during transfer of the first data stream, to utilize the maximum transfer rate to the greatest possible extent, so as to be able to minimize the follow-on accordingly. This may also be expedient in conceivable cases in which first detector data is purposefully acquired and provided for the first data stream, for example by reducing spatial resolution compared with corresponding information still contained in the second detector data. This is because the highest possible quality CT image data set is then provided even if the second detector data is no longer transferred.

Preferred selection options are described further below.

Where the second detector data is selected, it is compressed prior to transfer, and preferably even prior to temporary storage, using a two-stage compression method. The advantage of this two-stage compression method is that, on compression of the second detector data, the achievable compression rate is increased (so reducing transmission time) while at the same time limiting information loss. To this end, precompressed data is initially generated and fully compressed data is generated therefrom. The precompressed data is generated by compression of the second detector data using a lossy compression method. This means that information from the original detector data is no longer present after decompression. This is entirely acceptable if this lost data is not needed for later evaluation of CT images or merely results in immaterial impairment of image quality. On further compression of the precompressed data, however, data losses may have an altogether more serious effect. The fully compressed data is therefore generated using a lossless compression method.

The detector data in particular comprises one or more respective pixel values for each of a plurality of detector pixels of the X-ray detector, in particular of the detector pixel array. The detector data may comprise one or more respective pixel values for all detector pixels of the detector pixel array or for just a fraction thereof, for example for all detector pixels within a predefined continuous spatial region corresponding for example to the spatial structure of the detector pixel array as described above. The one or more pixel values may be stated as individual, scalar values or as inputs for one or more vectors, one or more matrices, or one or more higher-dimensional tensors, in particular if the CT device is a spectral CT device with a photon-counting X-ray detector.

The one or more pixel values, the precompressed data, and the fully compressed data comprise integer values, for example. The precompressed data is designated as such because it is the result of the lossy compression method but is further compressed using the lossless compression method.

The computer-implemented method according to one or more example embodiments of the invention implements a two-stage data compression scheme that precompresses the detector data using the lossy compression method and then further compresses the precompressed data using the lossless compression method. Consequently, the compression ratio achieved using the lossy compression method can be adapted or restricted to avoid significant information loss. Furthermore, the details of the lossy compression method may in part be physically motivated, for example, taking account of the noise level of the detector data, the Poisson distribution of the X-ray photons etc., so increasing the efficiency of the lossy compression method. The restricted compression rate may then be compensated by the subsequent lossless compression method. Known lossy and lossless compression methods may be used. Preferred lossy and lossless compression methods are described further below.

A system according to one or more example embodiments of the invention serves to operate a computed tomography device having a stationary portion and a portion that is rotatable relative to the stationary portion, wherein the rotatable portion has at least one X-ray detector, wherein the stationary portion has a computing means configured to process detector data, wherein the at least one X-ray detector has an assigned processing means for determining, on the basis of raw data from the at least one X-ray detector, detector data to be transferred via a communication path to the computing means and the communication path has a wireless communication link with a maximum transfer rate.

The system comprises the following components:
a selection unit for dividing the detector data of an imaging procedure into a first data stream, the first detector data of which is sufficient, after conclusion of raw data acquisition using the at least one X-ray detector, for reconstruction of an evaluatable CT image data set, and which has a data transfer rate corresponding at most to the maximum transfer rate, and into a second data stream comprising the remaining detector data as second detector data, according to at least one selection parameter predefinable by the control means,
a temporary storage unit for temporarily storing the second detector data of the second data stream in a temporary storage means, and
a transfer unit for direct real-time transfer of the first data stream via the communication path to the computing means and, after conclusion of raw data acquisition of the imaging procedure, for transfer of the second detector data via the communication path to the computing means,
a compression unit, designed to compress the second detector data, wherein, prior to transfer, precompressed data is generated by compression of the second detector data using a lossy compression method, and fully compressed data is generated from the precompressed data using a lossless compression method.

The functions of the system components have already been described above. The system is preferably designed to carry out a method according to one or more example embodiments of the invention. The units of the system are preferably software units that are executed on single computing means or in a system consisting of a plurality of computing means.

A control means according to one or more example embodiments of the invention for a computed tomography device comprises a system according to one or more example embodiments of the invention and/or is designed to carry out a method according to one or more example embodiments of the invention.

A CT device according to one or more example embodiments of the invention is in particular a photon-counting CT device, and comprises a control means according to one or more example embodiments of the invention.

The method may also be designated a computer-implemented method. One or more example embodiments of the invention can in particular be implemented in the form of a computer unit with suitable software. The computer unit can to this end have, for example, one or more cooperating microprocessors or the like. In particular, it may be implemented in the computer unit in the form of suitable software program parts. A largely software-based implementation has the advantage that computer units which are already in use can also straightforwardly be upgraded to operate in the manner according to one or more example embodiments of the invention via a software or firmware update. In this respect, the object is also achieved by a corresponding computer program product having a computer program that is directly loadable into a storage means of a computer unit and has program parts for carrying out all the steps of the method according to one or more example embodiments of the invention when the program is executed in the computer unit. In addition to the computer program, such a computer program product may optionally comprise additional elements such as for example documentation and/or additional components including hardware components, such as for example hardware keys (dongles etc.) for using the software.

A computer-readable medium, for example a memory stick, hard disk or other transportable or permanently installed data storage medium on which are stored the program parts of the computer program that can be read in and executed by a computer unit may be used for transport to the computer unit and/or for storage on or in the computer unit. CT images may here be captured, for example, through the output of corresponding control commands.

Further, particularly advantageous configurations and developments of one or more example embodiments of the invention are revealed by the dependent claims and by the following description, wherein the claims of one category of claim may also be further developed in a manner similar to the claims and passages of the description relating to another category of claim and in particular individual features of different exemplary embodiments or variants may also be combined to form new exemplary embodiments or variants.

For a specific preferred exemplary embodiment, provision may be made for the first data stream to comprise first detector data of at least partly reduced spatial resolution and/or spatial coverage relative to the total detector data, in particular also the second detector data alone. In exemplary embodiments that use such spatial sorting, detector data is therefore (initially) omitted and/or items of detector data are offset against one another, in order to reduce the spatial resolution. In the latter case, provision may also be made for the reduction in spatial resolution to be used purposefully to create the first detector data for the first data stream, this meaning that detector data of lower spatial resolution is sent first. A diagnostically usable CT image data set can already be determined from this data. In a second step, second detector data of higher spatial resolution may be delivered subsequently. Spatial resolution may be reduced for example by combining detector values from a group of adjacent pixels, for example 2×2 or 3×3 pixels, into a new pixel. This accordingly results in a reduction in the volume of data by a factor of four or nine respectively. Counting X-ray detectors have already become known, for example, the pixel size of which is of the order of 300 μm, such that a combination of nine pixels may still remain below a pixel size of 1 mm.

A further option for reducing the volume of data in spatial terms is the omission of specific data, for example a specific number of detector rows and/or detector columns, the detector data of which is subsequently delivered as second detector data. It was for example already proposed in the prior art for some use cases to transfer only a specific number of detector rows for each time step, so as not to exceed the maximum transfer rate. The remainder of the data had to be discarded. For the purposes of the present invention, this remaining detector data may be sorted into the second data stream and temporarily stored, such that it may be subsequently delivered.

In this connection, exemplary embodiments are also conceivable in which detector rows and/or columns are regularly omitted, for example only every nth row and/or every nth column is/are transferred. This opens up the option, which will be looked at in greater detail below, of the computing means making use of interpolation and/or extrapolation to estimate missing second detector data, namely data that has been lost or has been transmitted only at a later point in time, and to complete the detector data set once again at least in estimated form.

In this case, it is not necessary for the same spatial omission to be made for every time step in which the at least one X-ray detector is read out and new raw data arises and/or in which new detector data is processed. A specific configuration of the present invention may be provided, for example, for a predefined total spatial coverage of the total detector data to be broken down into at least two subgroups, wherein the raw data acquisition takes place in time steps, and in successive time steps in each case only the detector data of one of the subgroups, in accordance with a predefined order of subgroups, is selected into the first data stream. For example, detector rows with even row numbers and detector rows with uneven row numbers may be alternately transferred in time steps; it is also conceivable to change over between the upper and lower halves or the left and right halves. It goes without saying that more extensive subdivisions into subgroups are also possible. One example of such sampling is a type of “even/odd reading structure”, in which in every nth time step, i.e., reading, a different spatial structure is transferred and then, as a follow-on, the detector data omitted in each case is transferred in order to obtain the full information. This may improve interpolation operations which are to be carried out.

Time-based sorting may also be preferred for the purposes of the present invention. Provision may here therefore be made for the temporal resolution of the first detector data to be less than that of the total detector data. Provision may here, for example, also be made for the raw data acquisition to take place in time steps, wherein the first data stream comprises detector data only from every nth time step and the remaining detector data is assigned to the second data stream. In this case, only every nth reading is immediately transferred, with all the initially omitted detector data being sent as a follow-on, such that full temporal resolution is achieved. In this case, one embodiment provides for a sum and/or a mean to be determined and transferred over multiple, here n, time steps in the first data stream on transfer of the detector data of every nth time step.

Furthermore, for the purposes of the present invention frequency-based sorting is also preferred to take account of spatial and/or temporal variations. Provision may, for example, specifically be made for the processing means to comprise a transformation unit for determining the spatial frequency representation of the detector data, wherein only detector data with spatial frequencies that fulfill a spatial frequency criterion are sorted into the first data stream. It is thus proposed, for example, to apply methods such as those used to construct images in video streaming to use on a CT device, wherein only specific spatial frequencies are initially transferred and the missing information is only transferred at a point downstream. Selection may therefore be performed, for example, on the basis of the spatial frequencies arising (wavelets or indeed spatial frequencies). It is conceivable, for example, to verify using the spatial frequency criterion whether the spatial frequency lies below a spatial frequency threshold. In this case, initially only the low spatial frequencies are transferred, such that a coarser image is obtained before it is refined with the follow-on transfer of the second detector data. However, other selection operations are also preferred which are mapped by the spatial frequency criterion, such that, for example, successive spatial frequency intervals can be assigned alternately to the first detector data and the second detector data.

If detector data is omitted for the first data stream on a spatial and/or temporal and/or frequency basis, it may be preferred, as already indicated, for at least one part of the omitted detector data to be reconstructed by interpolation and/or extrapolation on reconstruction of a CT image data set from the detector data of the first data stream alone. This is expedient in particular also with regard to a breakdown in terms of spatial frequencies, since a usable, completed detector data set then immediately arises through interpolation on the receiver side, i.e., on the part of the computing means, in which data set estimated detector data can be replaced on the basis of follow-on data elements, i.e., second detector data, such that over time a complete detector data set is obtained. Interpolation may, for example, proceed by linear regression, wherein more complex regression methods, for example nonlinear regression, may also be used. Finally, it is also conceivable to use artificial intelligence-based methods, for example “super resolution”, or more generally methods which are also used for dynamic data construction when zooming in on internet maps and/or in the case of variable data rates when streaming videos.

In this case, it may also generally be noted at this point that it may be expedient to reconstruct and display a provisional CT image data set from the first detector data, optionally even online while it is being obtained. In this way, a user is also informed that, even in the event of abnormal termination or system failure, data of use to them is already available, improvement or expansion of which can then accordingly be tracked visually on arrival of the second detector data.

In a further option for selecting detector data, provision may be made for the CT device to be configured for spectral imaging and for the detector data to comprise partial data assigned to different energy parameter values, wherein the first data stream contains partial data only for some of the plurality of energy parameter values. For example, as has already been described, the at least one X-ray detector may be a counting detector and acquire the detector data for a plurality of energy threshold values as energy parameter values, wherein the first data stream comprises as first detector data only partial data of at least one, in particular the lowest, of the energy threshold values. In this case, spectral sorting is thus provided, wherein different partial data is transferred on immediate transfer of the first data stream than on follow-on transfer. In this way, the energy resolution capability of the at least one X-ray detector can be more extensively utilized by way of the follow-on information, i.e., the second detector data.

In this case, it goes without saying that combinations of these selection techniques are also conceivable. Thus, it is possible, for example, for spatial resolution for the partial data of the lowest energy threshold value to be reduced by way of the processing means, wherein the detector data arising is then sorted into the first data stream as first detector data. Since more photons are detected in the case of a low energy threshold value than in the case of a high energy threshold value, the noise of the raw data is less in the case of a low energy threshold value than in the case of a high energy threshold value. It is therefore often preferable, in practice, to spatially compress the data of the higher energy threshold value to a greater degree. The higher resolution detector data of the lowest energy threshold value and the detector data (optionally nonetheless of reduced spatial resolution) of further energy threshold values are initially temporarily stored as second detector data and transferred as a follow-on.

Selection may particularly advantageously be parameterizable. In this way, configurations of the method according to one or more example embodiments of the invention may provide that the selection unit undertakes division according to at least one selection parameter predefinable by a control means of the computed tomography device. For example, different capture protocols may have different selection parameters assigned to them that, for this capture protocol, on the one hand as far as possible use the maximum transfer rate in the first data stream and on the other hand provide the rough information desired for the specific imaging task with the first data stream and further desired information as the second detector data as a follow-on. In this respect, processing parameters for the processing means may in many cases moreover also already be generally assigned to the capture protocols, these processing parameters defining how the detector data is determined and thus the nature of the detector data to be determined.

Specifically, provision may in this case be made for the at least one selection parameter to be transmitted, with at least one processing parameter parameterizing the processing, from the control means to the processing means. In this case, at least one of the processing parameters, of which there is at least one, may be directly related to the selection. For example, at least one of the processing parameters, of which there is at least one, may be selected for the most extensive possible utilization of the maximum transfer rate in such a way that detector data for the first data stream arises in a suitable volume per unit time, for example by changing the spatial resolution. In other words, the desired information for each capture protocol is already known beforehand and may therefore be utilized for corresponding adjustment of the processing means, for example during “scan loading”, the components of the rotating portion of the computed tomography device also accordingly being prepared by the control means by parameterization.

In particular, this means that each selectable capture protocol predefining acquisition parameters for raw data acquisition and/or processing parameters for processing has the at least one corresponding selection parameter assigned to it and is transmitted to the processing means before raw data acquisition begins and is adjusted there. For example, the at least one selection parameter may be stored together with the at least one acquisition parameter and/or the at least one processing parameter in a lookup table of the control means.

It is particularly expedient for the at least one selection parameter and/or item of selection information describing division into the data streams also to be transmitted to the computing means, where, in a preparation step, the received detector data of the first data stream and of the second data stream is combined to form a detector data set suitable as input data for subsequent data processing processes of the computing means. In this way, the computing means, i.e., the image processor, may be informed as to the manner in which the detector data is divided into the first and the second data streams and the manner in which it will reach said means, such that a dedicated preparation step may be provided for downstream reconstruction steps for determining the final CT image data set, this dedicated preparation step generating a total detector data set sorted in the manner expected by the downstream reconstruction steps. In this way, computed tomography data sets may be determined in the same way, irrespective of how selection and transfer have proceeded. In other words, subsequent computing steps do not require any further knowledge of the “jumbled” data transfer of the detector data.

For transfers over the communication path, it is expedient to use a buffer that enables resending of a data packet of the first and/or second detector data in the event of faulty transfer. The provision of such buffers that allow resending is already known in principle in the prior art. As explained above, the first data stream is dispatched in real time, i.e., immediately, when new first detector data is available in each time step. In this case, the data packets are generally not all that large. However, this also means that only a brief time offset occurs should there be a transfer error.

In an expedient development, the buffer may take the form of a part of the temporary storage means reserved for this purpose. In this way, there is only a need to provide one storage means that provides the storage space as a temporary storage means for the second detector data and the buffer for data transfers via the communication path. The temporary storage means thus comprises a smaller portion of storage space therein that acts as a true buffer for resending in the event of direct transfer, and a larger portion of storage space in which the second detector data is saved and transferred once raw data acquisition has been concluded.

In one configuration, information preparing the next imaging procedure is transmitted to the rotating portion as early as during transfer of the detector data of the second data stream. In this way, efficient use of time can expediently be made and the next imaging procedure may be begun directly after conclusion of the transfer of the second detector data to the computing means. Such operation is always expedient if data transfer over the wireless data transfer path can proceed in parallel in both directions, and thus no portion of the maximum transfer rate is required for this preparation procedure, or a portion of the maximum transfer rate “remains free”.

In advantageous exemplary embodiments, provision may be made for the computing means to be configured to add additional information over and above the first detector data to the CT image data set at least with regard to the transferred part in the event of only partial transfer of the second detector data, which comprises the additional information, due to an error, in particular a system failure. This means that handling of the detector data on the computing means, in particular also with regard to the already discussed preparation step, is selected in such a way that, even in the event of partial transfer of the second detector data, the transferred part can still be put to meaningful use. For example, at least one part of the CT image data set may then be determined and represented at a higher spatial resolution and/or additional spectral information may be displayed at least in a part of the CT image data set. Corresponding detector data processing techniques are in principle already known in the prior art.

The detector data preferably corresponds to an image of an image sequence comprising the image and at least one further image. For each of the further frames, of which there is at least one, in each case further detector data is received, for example from the X-ray detector, and in each case further precompressed data is generated by compressing the respective further detector data using the lossy compression method. The fully compressed data is then preferably generated by compressing common precompressed data, which comprises or consists of the precompressed data for the image and the further precompressed data for the at least one further image.

The fact that the detector data corresponds to the image may in this case be understood in particular to mean that it is or has been generated by the X-ray detector during the image. In other words, while individual images are precompressed separately using the lossy compression method, the precompressed data from two or more images, in particular from a stack of images, is jointly compressed using the lossless compression method. This improves the overall efficiency and/or speed of the method.

The lossless compression method is preferably an entropy coding method, for example the lossless compression method is a method for asymmetric numeral systems (ANS). Such implementations are particularly advantageous since the lossy compression method may reduce the entropy of the data compressed using the entropy coding method. This increases the compression ratio of the lossless compression method and consequently also the total compression ratio.

The detector data preferably comprises a respective pixel value for each of a plurality of detector pixels of the X-ray detector and the lossy compression method comprises pixel-by-pixel lossy compression. To this end, a respective precompressed pixel value is calculated for each detector pixel of the plurality of detector pixels. This proceeds in particular as a function of the respective pixel value. For pixel-by-pixel lossy compression, it should be borne in mind that there may be a one-to-one correspondence between the pixel value of a specific detector pixel and the corresponding precompressed pixel value. One-to-one correspondence is preferred if a pixel is defined solely by way of the spatial coordinates. However, a lossy pixel value of a detector pixel may also depend on the pixel value of a previous reading (i.e., of detector readout of an earlier point in time). Lossy compression preferably also comprises difference coding (for the same pixel but an earlier point in time). It is preferred in this case for the pixel-by-pixel lossy compression for each detector pixel of the plurality of detector pixels to comprise calculation of the precompressed pixel value for the respective detector pixel using a predefined compression function, the argument of which is dependent on the respective pixel value.

In pixel-by-pixel lossy compression, for example, for each detector pixel of the plurality of detector pixels, the precompressed V pixel value is calculated for the respective detector pixel using a predefined lossy compression function, the argument of which is dependent on, for example comprises or consists of the respective pixel value. Preferably, the lossy compression function is dependent on the pixel value and on the temporally previous pixel value (at the same location). Below, the lossy compression function is also designated compression function.

The compression function may also be designated assignment function. The compression function may, for example, be an assignment function as described in above-mentioned document U.S. Pat. No. 9,078,625 B2.

For example, a codomain of the compression function may be a set of integers, in particular given by [N_0, N]∩N, wherein N_0 is a predefined positive integer or zero and N is a predefined positive integer greater than N_0. In particular, the cardinality of the codomain is smaller than the cardinality of a domain of the compression function, which likewise corresponds to a set of integers.

For example, the cardinality of the coding region is provided by a predefined bit depth, designated m. In particular, the cardinality of the coding region may be 2m. The codomain may for example be provided by {0, 1, 2, . . . , 2m−1}. The bit depth m may for example be an integer between 5 and 15, for example 8 to 15.

It is noted in relation to the following that, in the case of lossy compression, there is one-to-one correspondence between the pixel value of a specific detector pixel and the corresponding precompressed V pixel value. The detector pixels will be looked into below with regard to lossless compression. The term “V pixel value” is used here as a way of identifying a precompressed pixel value in connection with detector pixels. This V pixel value is part of the precompressed data after lossy compression.

Lossless compression preferably involves the following steps for each V pixel value of the precompressed data:
receiving historical V pixel values for each surrounding pixel within predefined historical surroundings of the respective V pixel value of a detector pixel;
calculating a predictor value for the respective V pixel value as a function of the historical V pixel values of the surrounding pixels of the respective V pixel value of the detector pixel and independently of this V pixel value of the respective detector pixel; and
generating the fully compressed data as a function of the V pixel values for the respective detector pixel and of the predictor value for the respective V pixel value.

It is preferable in this case for these steps to be carried at least in part during transmission of the first data stream.

According to this preferred embodiment, for each V pixel value of the plurality of detector pixels, lossless compression thus involves receiving historical V pixel values of each surrounding pixel within predefined historical surroundings of the respective detector pixel. “Historical” thus means here those pixel values or surroundings that have been previously processed during a compression procedure. In the case of pixel-by-pixel compression, pixels that have already been processed in the context of this compression would thus be considered “historical”. For each V pixel value of the detector pixels of the plurality of detector pixels, a predictor value is then calculated as a function of the historical V pixel values of the surrounding pixels of the respective detector pixel, in particular all surrounding pixels of the respective detector pixel, and independently of the V pixel value of the respective detector pixel. The fully compressed data, in particular a corresponding part of the fully compressed data, is generated as a function of the V pixel values of the precompressed data for the respective detector pixel and of the predictor value for the V pixel value of the respective detector pixel. It should again be noted that “V pixel value of a detector pixel” here means the values of the precompressed data that correspond to precompressed values of real detector pixels, the values of which have been compressed in a lossless manner.

In particular, the predictor value may be used to estimate the precompressed V pixel value for the respective detector pixel on the basis of the historical V pixel values. This improves lossy pixel-based compression efficiency.

The historical surroundings are preferably uniquely predefined for each V pixel value for the detector pixels of the plurality of detector pixels, in particular in the form one or more rules. In this case, all the surrounding pixels of the historical surroundings have undergone pixel-by-pixel lossy compression before lossless compression is applied to the V pixel value respectively under consideration, enabling the historical surroundings to be designated “historical”. In particular, the historical surroundings do not consist of the respective V pixel value under consideration itself. Consequently, the respective precompressed V pixel values of the historical surroundings are straightforwardly available during the decompression process. The historical surroundings may consist of surrounding pixels comprising pixels from an earlier image. Alternatively or in addition, the historical surroundings may consist of surrounding pixels that originate from the same image as the respective pixel under consideration. Consequently, the one or more rules that define the historical surroundings contain an initialization rule for an initial pixel. In this case, the historical surroundings may, for example, be empty and the respective historical pixel values may be set to zero or to other values predefined for this case.

For a specific V pixel value for a specific detector pixel in a specific image, the surrounding pixels may comprise V pixel values from adjacent pixels of the specific detector pixel. The historical surroundings may, for example, consist of a continuous region of detector pixels that excludes the given detector pixel. In other words, the historical surroundings are a predefined vicinity of the given detector pixel, related, however, to the V pixel values of the precompressed data. This is not necessarily the case, however. In particular, the historical surroundings may also comprise surrounding pixels, the V pixel values of which are presumably correlated with the V pixel value of the given detector pixel. This may be the case, for example, with pixels in similar parts of the detector array or with pixels within the same subarray of the detector array etc. The number of surrounding pixels within the historical surroundings may be equal to one or greater than one. In some implementations, the historical surroundings of a specific pixel may even consist of the same pixel in the previous (readout) image.

The argument of a predictor value (optionally, however, also of a compression function) is preferably dependent not only on the respective V pixel value but also on the historical V pixel values of the surrounding pixels of the respective detector pixel. For example, the argument of the predictor value or of the compression function may comprise or consist of the respective V pixel value and the historical V pixel values of the surrounding pixels of the respective detector pixel. In this way, during pixel-by-pixel compression, the correlation between different pixels of the detector array can be further utilized to improve the efficiency of the lossy compression method.

The predictor value is preferably calculated as the weighted sum or weighted average of the historical V pixel values of the surrounding pixels of the respective detector pixel. This provides a particularly simple way of generating the predictor value with very limited computational effort. Furthermore, due to Gauss' law of error propagation, the noise of a predictor generated in this way is less than that of the V pixel values in question.

Preferably, the predictor value is calculated by application of a trained machine learning model (MLM) to input data comprising or consisting or the historical V pixel values of the surrounding pixels of the respective detector pixel. In particular, training based on training data may enable the MLM to adapt to new circumstances and to recognize and extrapolate patterns. In general, the parameters of an MLM can be adapted or updated by training. Supervised training, semi-supervised training, unsupervised training, reinforcement learning and/or active learning may in particular be used. Representation learning, which is also known as feature learning, can furthermore be used. In particular, the parameters of the MLMs can be iteratively adapted by a plurality of training steps. In particular, a given loss function, also known as a cost function, may be minimized in the context of training. In the context of training an artificial neural network (ANN), it is in particular possible to use the backpropagation algorithm.

An MLM may in particular encompass an “artificial neural network” (ANN), a support vector machine, a decision tree and/or a Bayesian network and/or the MLM may be based on k-means clustering, Q-learning, genetic algorithms and/or association rules. An ANN may in particular be or comprise a deep neural network, a convolutional neural network, or a deep convolutional neural network. Moreover, an ANN may be an adversarial network, a deep network and/or a generative network.

In one embodiment of the method, it is preferred, for each V pixel value of the detector pixels of the plurality of detector pixels,
to calculate a precompressed predictor value on the basis of the predictor value for the V pixel value of the respective detector pixel;
to calculate a difference value between the (precompressed) V pixel value for the respective detector pixel and the precompressed predictor value for the respective detector pixel; and
to generate the fully compressed data as a function of the difference values for the plurality of detector pixels.

It is preferred in this case for
the predictor value for the V pixel value of the respective detector pixel to be calculated as a weighted sum of the historical V pixel values; or
the predictor value for the V pixel value of the respective detector pixel by application of a trained machine learning model to input data comprising the historical V pixel values.

A precompressed predictor value is thus preferably calculated on the basis of the predictor value for the V pixel value for the respective detector pixel. A difference value is calculated between the (precompressed) V pixel value for the respective detector pixel and the precompressed predictor value for the respective detector pixel. The fully compressed data, in particular the respective part of the fully compressed data, is generated as a function of the difference values, in particular of offset difference values calculated for the plurality of V pixel values relating to the detector pixels.

The precompressed predictor value is calculated, in particular, on the basis of the predictor value in the same manner or using the same calculation as for the calculation of the precompressed V pixel value. In particular, the precompressed predictor value may be calculated by application of the compression function to the predictor value or, in corresponding implementations, to the predictor value and the historical V pixel values for the respective detector pixel.

Since the historical V pixel values potentially correlate with the V pixel value of the respective detector pixel, the precompressed predictor value may be regarded as an estimate or prediction of the (precompressed) V pixel value. The difference between the two values is thus potentially small. This improves the efficiency of the pixel-based lossless compression.

The precompressed data (V pixel values) may contain the value (D(x)+2m)mod 2m within the codomain of the compression function, for example for each of the plurality of detector pixels. Therein, m is the predefined bit depth, x is the V pixel value of the respective detector pixel and D(x)=L(x)−L(Px) is the difference value between the (precompressed) V pixel value for the respective detector pixel and the precompressed predictor value for the respective detector pixel. Therein, L designates the compression function and Px the predictor value for the respective detector pixel. By adding and the term 2m to the difference value, it is ensured that D(x)+2m is positive and mod 2m ensures that D(x)+2m)mod 2m lies within the codomain of the compression function. The value (D(x)+2m)mod 2m can be designated the offset difference value.

The predictor values can then be used for decompression. In practice, it may even be the case that the predictor values are needed if it is to be possible to carry out lossless decompression at all. They supply additional information about the fully compressed data that make lossless decompression of the fully compressed data quicker or simpler. The (optionally precompressed) predictor values may in particular be prepared during sending of the online data during full compression of the (offline) data (second detector data) and sent immediately thereafter, be sent together with the offline data or be sent after the offline data has been sent. What is important is that they are available for decompression of the fully compressed data.

A further aspect of one or more example embodiments of the invention provides a computer-implemented method for compression and decompression of data from a computed tomography device. A computer-implemented method for compressing the data from the CT device according to one or more example embodiments of the invention is in this case carried out by a first data processing system.

In a first alternative, the fully compressed data is transferred from the first data processing system to a second data processing system, in particular via a transmission link of the CT system, for example a slip ring of the CT system, or the fully compressed data from the first data processing system is stored in a storage means (temporary storage means) and the fully compressed data is read out from the storage means by the second data processing system.

In one embodiment of the method, once the compressed second detector data has been transferred, it is preferred: for the precompressed data to be restored on the basis of the fully compressed data by reversal of the lossless compression method and for decompressed data to be generated on the basis of the restored precompressed data by reversal of the lossy compression method.

It is preferred in this case that
reversal of the lossless compression method comprises lossless decompression, wherein a respective (decompressed) V pixel value is calculated for each detector pixel of the plurality of detector pixels,
lossless decompression for each V pixel value for each detector pixel of the plurality of detector pixels comprises receiving the recovered historical V pixel values of each surrounding pixel within the historical surroundings of the respective detector pixel, recalculating the predictor value for the respective detector pixel as a function of the recovered historical V pixel values of the surrounding pixels of the respective detector pixel, and calculating the respective V pixel value as a function of the recovered precompressed data and the recalculated predictor value for the respective detector pixel.

The precompressed data is thus preferably restored by the second data processing system on the basis of the fully compressed data by reversal of the lossless compression method, in particular the entropy coding method, for example, the ANS method. The decompressed data is generated by the second data processing system on the basis of the restored precompressed data by reversal of the lossy compression method.

In a second alternative, the fully compressed data from the first data processing system is stored on the memory device and the fully compressed data is read out from the memory device by the first data processing system at a later point in time. The precompressed data is then restored by the first data processing system on the basis of the fully compressed data by reversal of the lossless compression method, in particular the entropy coding method, for example, the ANS method. The decompressed data is generated by the first data processing system on the basis of the restored precompressed data by reversal of the lossy compression method.

In a preferred method for compression and decompression of data from the computed tomography device, reversal of the lossy compression method comprises pixel-by-pixel lossy decompression, wherein a decompressed pixel value corresponding to each detector pixel of the plurality of detector pixels (from the V pixel values) is calculated.

The pixel-by-pixel lossy decompression is in particular a reversal of the pixel-by-pixel lossy compression and may for example involve correspondingly applying a reversal of the compression function.

In a preferred method for compression and decompression of data from the computed tomography device, the computer-implemented method for compressing the data from the computed tomography device is carried out such that the fully compressed data is generated as a function of the (precompressed) V pixel value for the respective detector pixel and the predictor value for the V pixel value of the respective detector pixel, as described above.

Carrying out lossless decompression for each V pixel value for each detector pixel of the plurality of detector pixels comprises receiving fully compressed data, recalculating the predictor value for the V pixel value of the respective detector pixel as a function of the fully compressed data, and calculating the respective (now decompressed) V pixel value as a function of the fully compressed data for the V pixel value and the recalculated predictor value for the respective detector pixel.

In particular, the pixel order on lossy decompression is identical to the pixel order on lossy compression. Thus, if the decompressed pixel value is to be calculated for a specific detector pixel, the decompressed pixel values for the surrounding pixels within the historical surroundings or, in other words, the historical pixel values, have already previously been calculated and are available.

In a preferred method for compression and decompression of data from the computed tomography device, the computer-implemented method for compressing data from the computed tomography device is carried out such that the precompressed data is generated as a function of the difference values for the plurality of detector pixels, in particular the offset difference values, as described above.

In one embodiment of the method, it is preferred that
a further precompressed predictor value is calculated on the basis of the recalculated predictor value;
for each V pixel value for each detector pixel of the plurality of detector pixels, the difference value for the V pixel value for the respective detector pixel is recovered on the basis of the recovered precompressed data, a cumulative value of the recovered difference value for the V pixel value for the respective detector pixel and the further precompressed predictor value for the respective detector pixel is calculated, and the respective (now decompressed) V pixel value is calculated as a function of the cumulative value.

It is preferred in this case that
the respective (now decompressed) V pixel value is calculated by applying a reversal of the lossless compression function to an argument that is dependent on the respective cumulative value.

A further precompressed predictor value is thus preferably calculated on the basis of the recalculated predictor value, for example by application of the reversal of the compression function to the recalculated predictor value. For each V pixel value for each detector pixel of the plurality of detector pixels, the difference value for the V pixel value for the respective detector pixel, in particular the offset difference value mod 2m, is restored on the basis of the fully compressed data. A cumulative value of the recovered difference value for the V pixel value for the respective detector pixel and the further precompressed predictor value for the respective detector pixel is calculated or a cumulative value of the recovered offset difference value mod 2m and the further precompressed predictor value. The respective (now decompressed) V pixel value is determined as a function of the cumulative value. In particular, calculation of the cumulative value reverses the calculation of the difference value during compression. For example, the respective (now decompressed) V pixel value is calculated by applying a reversal of the compression function to an argument that is dependent on, in particular consists of or contains, the respective cumulative value.

The following is particularly advantageous for practical implementation:
a) The predictor value is calculated as soon as all the historical pixels (that contribute to the predictor value) have been decompressed. The first predictor of the first pixel does not require a historical pixel.
b) When a current pixel is decompressed in a lossless manner, the difference
D=(V pixel value predictor value), or D=(V pixel value−predictor value+2m) should this difference D be negative, is initially obtained.
c) The V pixel value can therefore be calculated as V pixel value=D+predictor value mod 2m.
d) Finally, the actual pixel value can be calculated by lossy decompression from the V pixel value (some of the historical pixels, preferably pixel values from the preceding detector readout, are generally used for this purpose).
e) As a result, the quantity of available historical pixels is increased and the predictor value of the next pixel may be calculated in a).

According to a preferred embodiment of the method, the second detector data is compressed before it is temporarily stored. It is preferred in this case for the second detector data to be stored together with predictor values determined therefrom. This has the advantage that the “offline data set” is available in collected form.

If the second data processing system generates the decompressed data, a reconstructed CT image that obtains the decompressed data from the second data processing system is preferably generated by the second data processing system or by a third data processing system. If the first data processing system generates the decompressed data, the reconstructed CT image that obtains the decompressed data from the first data processing system is generated, for example, by the first data processing system or by the third data processing system.

In particular, a known CT reconstruction method may be used to generate the reconstructed CT image as a function of the decompressed data.

In particular, the data processing device may comprise one or more computers, one or more microcontrollers and/or one or more integrated circuits, for example one or more application-specific integrated circuits (ASIC), one or more field-programmable gate arrays (FPGA) and/or one or more systems on a chip (SoC). The data processing device may also contain one or more processors, for example one or more microprocessors, one or more central processing units (CPU), one or more graphics processing units (GPU), and/or one or more signal processors, in particular one or more digital signal processors (DSP). The data processing device may also comprise a physical or virtual cluster of computers or of others of the stated units.

In various embodiments, the data processing device comprises one or more hardware and/or software interfaces and/or one or more memory units.

A memory unit may a volatile data memory, for example of a dynamic random access memory (DRAM) or static random access memory (SRAM), or of a non-volatile data memory, for example of a read-only memory (ROM), programmable read-only memory (PROM), an erasable programmable read-only memory (EPROM), an electrically erasable programmable read-only memory (EEPROM), a flash memory or flash EEPROM, a ferroelectric random access memory (FRAM), a magnetoresistive random access memory (MRAM), or a phase-change random access memory (PCRAM).

According to a preferred embodiment of the system, the compression unit (also referred to as a compression device) comprises a first data processing system which is suitable for carrying out a computer-implemented method for compression and decompression of data from a CT device according to one or more example embodiments of the invention, which method in particular comprises the described generation of decompressed data.

According to a preferred embodiment of the system, the latter comprises a decompression unit (also referred to as a decompression device) with a second data processing system. The first data processing system and the second data processing system are suitable for together carrying out a computer-implemented method for compression and decompression of data from a computed tomography device according to one or more example embodiments of the invention. This may be understood as follows: the first data processing system is set up to carry out the computer-implemented method for compressing the data from the computed tomography device and to transfer the fully compressed data to the second data processing system or to store the fully compressed data in the storage means. The second data processing system is set up to receive the fully compressed data from first data processing system or to read it out from the storage device. The second data processing system is furthermore set up to carry out the remaining steps of the computer-implemented method for compression and decompression of data from a computed tomography device, in particular to reverse the lossless compression method and to reverse the lossy compression method.

According to a preferred embodiment of the method, information preparing the next imaging procedure is transmitted to the rotating portion as early as during transfer of the compressed second detector data.

It is preferred in this case that second detector data of the next imaging procedure is compressed in this context.

In one embodiment of the method, it is preferred that, prior to being compressed, the second detector data is subdivided into sub-data sets and precompressed data is then correspondingly generated from the sub-data sets by separate compression of the sub-data sets using the lossy compression method, and fully compressed data is generated from the precompressed data using a lossless compression method, wherein individual compressed data sets are preferably generated.

It is preferred in this case that individual predictor values are calculated for each data set.

A preferred system comprises a decompression unit that is designed to restore the precompressed data on the basis of the fully compressed data by reversal of the lossless compression method and to generate decompressed data on the basis of the restored precompressed data by reversal of the lossy compression method.

AI-based methods (AI=“artificial intelligence”) are preferably used for the method according to one or more example embodiments of the invention. Artificial intelligence is based on the principle of machine-based learning and is typically carried out with an algorithm that is capable of learning and has been appropriately trained. Machine-based learning often also known as “machine learning”, this also encompassing the principle of “deep learning”.

Components of one or more example embodiments of the invention preferably take the form of a “cloud service”. Such a cloud service serves to process data, in particular using artificial intelligence, but may also be a service based on conventional algorithms or a service in which interpretation is provided in the background by humans. In general, a cloud service (hereinafter also denoted “cloud” for short) is IT infrastructure in which for example storage space or computing power and/or application software is provided across a network. Communication between the user and the cloud here proceeds by way of data interfaces and/or data transfer protocols. In the present case, it is particularly preferred for the cloud service to provide both computing power and application software.

In the course of a preferred method, data that is obtained in course of one or more example embodiments of the invention is provided via the network to the cloud service. The latter comprises a computer system that typically does not comprise the user's local computer. The method may here be implemented using a command constellation in a network. The data calculated in the cloud is subsequently sent back via the network to the local computer of the user.

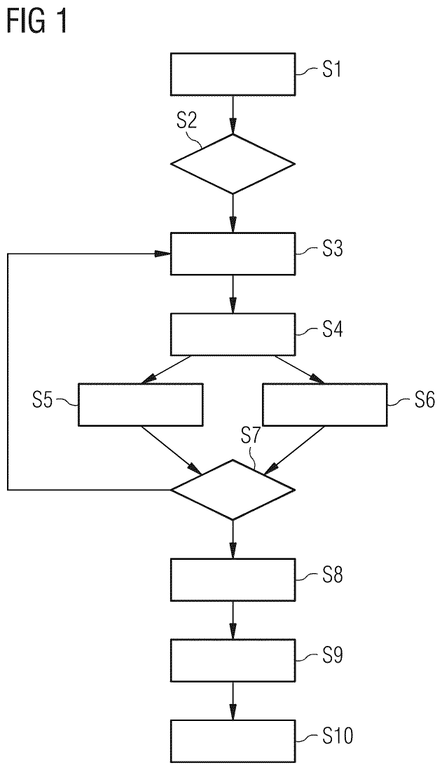

FIG. 1 shows a general flowchart of a method according to one or more example embodiments of the invention for operating a computed tomography device on performance of an imaging procedure on an object, in this case a patient. The patient is in this case placed in a gantry of the computed tomography device in such a way that at least one capture assembly of the computed tomography device, comprising an X-ray emitter and an X-ray detector, may be rotated around the patient, or more precisely a capture region of the patient. The capture assembly is therefore part of a rotatable portion of the computed tomography device that can be rotated relative to a stationary portion, for example the gantry. A computing means serving as an image processor of the computed tomography device together with the control means that controls the operation of the computed tomography device are also stationary, wherein the computing means may be part of the control means. A communication path, which in the present case includes a wireless communication link to ensure optimal rotational mobility, is provided in order to be able to transfer information for driving the components of the rotatable portion of the computed tomography device and to transfer detector data acquired using the at least one X-ray detector and provided by a processing means, which is also part of the rotatable portion, to the computing means. The wireless communication link has a maximum transfer rate, which means that a data stream with a data transfer rate that is greater than the maximum transfer rate cannot be transferred unbuffered in real time via the wireless communication link and thus via the communication path.

Nevertheless, it is a requirement for CT devices that, even in the event of system failure or other termination of measurement, detector data that has already been acquired must be persistently stored and usable for reconstructing a diagnostically usable computed tomography image. The method described below makes it possible to meet this requirement and at the same nevertheless to provide additional, more in-depth information.

In a step S1, the control means sends control information via the communication path to the drivable components of the rotatable portion in order to prepare for raw data acquisition using the at least one detector. The control information in this case comprises on the one hand processing parameters that indicate how the raw data from the at least one X-ray detector is to be processed to yield detector data that is to be transferred to the computing means. However, on the other hand, the control information also includes selection parameters for parameterizing a selection unit of the processing means, in which the detector data arising during each time step is stored in a first data stream that is to be immediately transferred to the computing means, and in a second data stream, the second detector data of which is stored in a temporary storage means, which in this case is integrated in the processing means, and only transmitted to the computing means once the first data stream and raw data acquisition are complete. The processing parameters and selection parameters are here coordinated with one another, in particular also such that the data transfer rate of the first data stream approximates as closely as possible to the maximum transfer rate of the wireless communication link. Selection information describing the division into data streams is also transmitted to the computing means, such that detector data received there in a preparation step can ultimately be sorted (and optionally decompressed) continuously such that it can be processed by subsequent reconstruction steps and so delivered in a desired format. The reconstruction steps thus do not themselves require any knowledge of the specific data transmission described here.

The processing parameters and selection parameters together with the selection information may here be assigned a selected capture protocol for the imaging procedure, for example by way of a lookup table in the control means. The assigned control information here conventionally also comprises further acquisition parameters for raw data acquisition.

In a step S2, raw data acquisition then begins, according to which, for each time step in which the at least one X-ray detector measures raw data, this raw data is processed by the processing means in step S3 in order to determine detector data.

In a step S4, the selection unit of the processing means divides the incoming detector data stream into the first data stream and the second data stream, for which purpose selection criteria, for example parameterized by way of the selection parameters, may be used. Examples of such selection criteria and how it is possible to remain below the maximum transfer rate are discussed in greater detail below.

Steps S5 and S6 then run in parallel. According to step S5, the first data stream comprising the first detector data, in particular one data packet for each time step, is directly transferred onward to the computing means via the communication path. Buffering by way of a small buffer, which permits individual data packets to be resent, is carried out if need be in the event of a transfer error. Real-time transfer is thus ensured. Step S5 may be carried out by a transfer unit (also referred to as a transfer device) of the processing means. In parallel thereto, in step S6, the second data stream comprising the second detector data is forwarded by way of a temporary storage unit to the temporary storage means where it is temporarily stored. The small buffer mentioned in relation to step S5 may here moreover also be provided as a small portion of the temporary storage means.

In a step S7, it is then verified whether there are any further time steps to follow or whether raw data acquisition is complete. If there are any further time steps to follow, the procedure is continued as in step S3, while if raw data acquisition is complete, the second detector data from the temporary storage means is transferred to the computing means unit as a follow-on by way of the transfer unit in step S8 once the transfer of the first data stream has also been completed. The maximum transfer rate is here ideally also utilized to the greatest possible extent.

In exemplary embodiments, it is conceivable for control information to be sent to the components of the rotating portion in preparation for the next imaging procedure as early as during step S8 providing this would not slow and/or disrupt the transfer of the second detector data. The small buffer may, of course, also be used for the second detector data.

In step S9, as already mentioned, the computing means carries out the preprocessing step by sorting received detector data using the selection information and converting it into a format that the following reconstruction steps expect for the detector data. In step S10, these following reconstruction steps utilize the appropriately prepared detector data for reconstruction of a CT image data set.

It should be noted in this respect that steps S9 and S10 can at least in part also be carried out as early as during transfer of the detector data. For example, once the first detector data has been obtained, it is conceivable initially to reconstruct and display a provisional CT image data set which, when the second detector data is obtained after conclusion of the transfer or even during the transfer, is then incrementally updated and improved on the basis of the further information.

It should additionally be noted in this respect that, when a CT image data set is determined solely from the first detector data, if the maximum transfer rate is observed by omission in spatial, temporal and/or spatial frequency terms, interpolation may be performed, for example by linear regression, as well as more complex methods, for example using artificial intelligence.

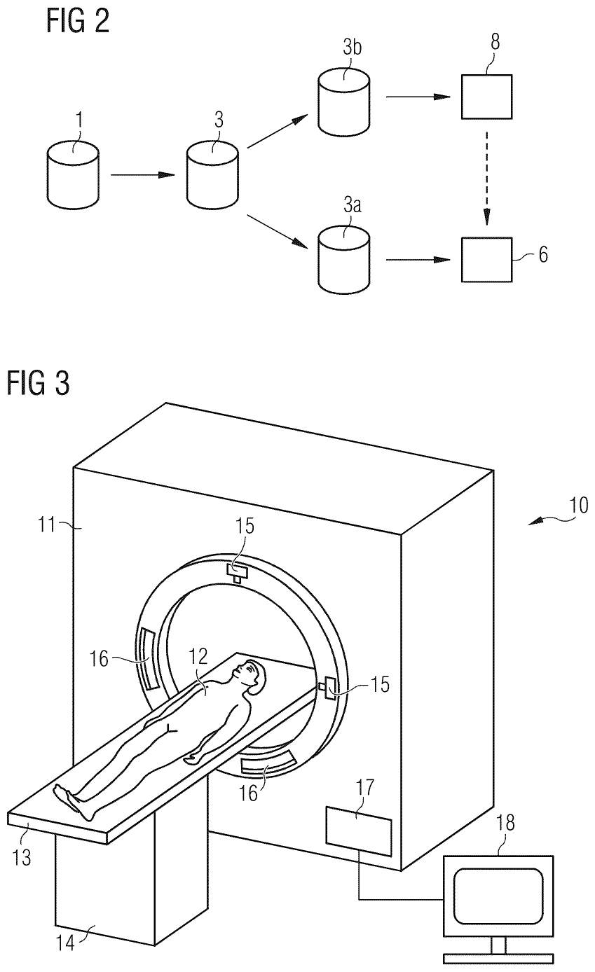

By way explanation, FIG. 2 shows the data flow according to the method of FIG. 1, where raw data 1 initially arises on the at least one detector and is processed in the processing means (arrow) to yield a totality of detector data 3. In the selection unit of the processing means, the detector data 3 is divided according to the arrows into first detector data 3a of the first data stream and second detector data 3b of the second data stream. The first detector data 3a is directly transferred (arrow) to a computing means 6, while the second detector data 3b is forwarded (arrow) as the second data stream into the temporary storage means 8, from where it may then likewise be transferred according to the dashed arrow to the computing means 6 after conclusion of raw data acquisition.

Depending on the specific imaging task, division (arrows) into the first data stream and the second data stream may be carried out on the basis of spectral sorting, temporal sorting, spatial sorting and/or spatial frequency-based sorting. If a counting detector, which can also quantify the energy of individual X-ray quanta, is used as preferred, raw data may be measured, for example, for a plurality of energy threshold values, wherein only detector data 3 of some of the energy threshold values, in particular the at least one lowest energy threshold value, may be selected into the first data stream; the detector data 3 from other energy threshold values is sorted into the second data stream. In the case of temporal sorting, it is possible to transfer only the readout result from the nth time step. A number of configurations are conceivable in the case of spatial sorting. On the one hand, detector data 3 with a lower spatial resolution may be determined and immediately transferred as the first detector data 3a, while high-resolution detector data 3 is transferred as a follow-on as the second detector data 3b. Spatial sorting may, however, also be performed by provisionally omitting detector data 3, such that for example only a selection of available image rows need be selected into the first data stream. In this case, the subgroups of a total spatial coverage selected for the first data stream need not be identical for each time step, but may instead also, for example, alternate from time step to time step or follow a predefined subgroup order.

With regard to spatial frequencies, the processing means may have a transformation unit for determining a spatial frequency representation of the detector data 3, such that for example only the detector data 3, the spatial frequency of which is below a spatial frequency threshold may be selected into the first data stream. Transfer methods of the kind used in prior art video streaming may also be used, for example a breakdown on the basis of the occurring spatial frequencies (wavelets), such that a usable detector data set is obtained by interpolation on the receiver side, which data set may be combined with the follow-on data elements, i.e., the second detector data 3b, to form a complete data set. As already mentioned, interpolation may be performed, for example, by simple linear regression, but more complex approaches, for example those using artificial intelligence are also conceivable, one example of which is known as “super resolution”.

In a very specific example, the X-ray detector may for example have a detector area of 2752×288 pixels, wherein two energy thresholds are used in the counting X-ray detector. The resultant volume of data is significantly too large for direct transfer. Detector data 3 is therefore determined in the processing means with a lower spatial resolution, for example 1376×144 pixels, for both energy threshold values. This first detector data 3a is selected into the first data stream and immediately transferred. The spatially high-resolution detector data is additionally available as second detector data 3b in the temporary storage means 8 and may be transferred as a follow-on. It may in this case be sufficient to transfer the high spatial resolution detector data 3b for just one of the energy threshold values. The detector data 3 is then available on the computing means 6 in such a way that detector data 3 of the lower energy threshold value is present once at high and once at low spatial resolution, while detector data of the upper energy threshold value is, however, present only at the reduced spatial resolution.

Variations are, of course, also conceivable, in which for example only partial data of the one energy threshold value is initially transferred and the like.

FIG. 3 shows a schematic outline of a CT device 10 according to one or more example embodiments of the invention that is configured to carry out the method according to one or more example embodiments of the invention.

The computed tomography device 10 (CT device 10 for short) comprises a gantry 11 that has a central opening into which a patient 12 may be advanced by way of a patient couch 13 of a patient table 14. The rotating portion comprises two capture assemblies, here shown only indicatively, each having an X-ray emitter 15 and a counting X-ray detector 16. The device is therefore a dual-source CT device and/or a biplane CT device 10 that is configured for spectral imaging. Operation of the computed tomography device 10 is controlled by a control means 17 that may also have an assigned display means 18 and may comprise the computing means 6.

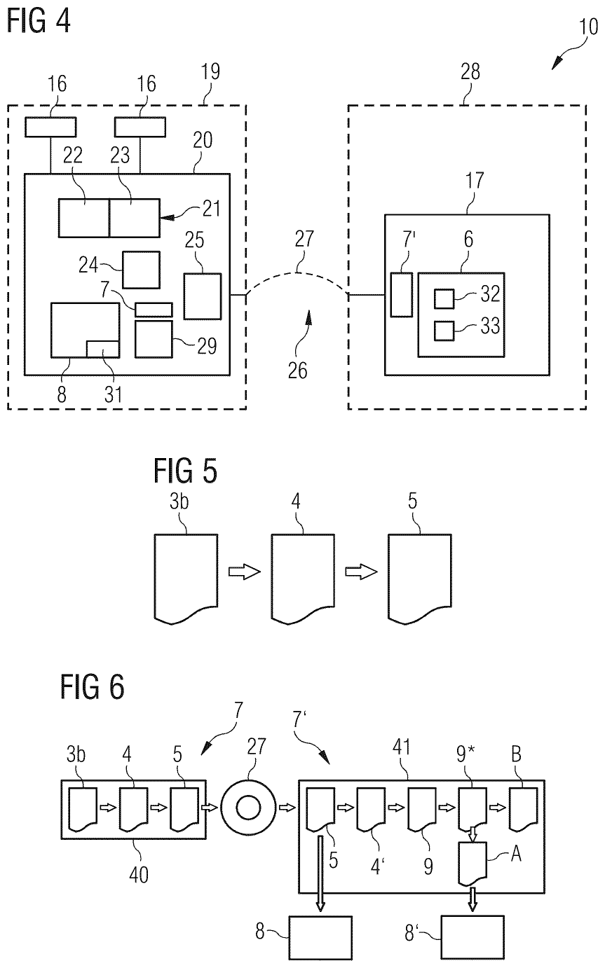

The functional structure of the computed tomography device 10 is shown once again in simplified form in FIG. 4.

As mentioned, the CT device 10 comprises a rotating portion 19, of which both X-ray detectors 16 are also a part. These deliver their raw data to the processing means 20 that comprises the selection unit 24 in addition to processing units 21—for example comprising the transformation unit 22 and a spatial resolution reduction unit 23. Thus, while the raw data is being processed by the processing units 21 according to step S3 to yield the detector data 3, the detector data 3 is being selected and divided into the data streams according to step S4 in the selection unit 24. The first data stream is forwarded to a transfer unit 25, which transfers the first detector data 3a directly via the communication path 26 with the wireless communication link 27 to the control means 17 in the stationary portion 28, and thus also to the computing means 6, which is part of the control means 17. In other words, the transfer unit 25 carries out step S5.

The second data stream is forwarded to a temporary storage unit 29 that temporarily stores the second detector data 3b in the temporary storage means 8 of the processing means 20 according to step S6. It should be noted at this point that part of the temporary storage means 8 may also be reserved as a buffer 31 for resending data packets for the transfer unit 25.

The system comprises a compression unit 7 according to one or more example embodiments of the invention, which is here arranged on the temporary storage unit 29 and which compresses the data stored in the temporary storage unit 29. Once transferred, the transferred data, if it was compressed, is decompressed again. This is performed with the decompression unit 7′.

Once raw data acquisition and transfer of the first data stream to the computing means 6 are concluded, the transfer unit 25 may also transfer the second detector data 3b via communication path 26 to the computing means 6 (step S8).

The computing means 6 may, for example, have a preparation unit 32 for carrying out preparation step S9 and a reconstruction unit 33 for carrying out step S10, i.e., reconstruction of a CT image data set.

FIG. 5 shows a flow chart of a computer-implemented method for compressing data from the computed tomography device 10 according to one or more example embodiments of the invention, in which second detector data 3b from the X-ray detector of the computed tomography device 10 is received. Precompressed data 4 is generated by compression of the second detector data 3b using a lossy compression method. Fully compressed data 5 is generated as a function of the precompressed data 4 using a lossless compression method, for example an entropy coding method such as an ANS method.

The system comprises a compression unit 7 according to one or more example embodiments of the invention. On the basis of FIGS. 3 and 6, a first data processing system 40 of the compression unit 7 is comprised by the CT scanner 11 and in particular arranged in a rotating part of the CT scanner 11. The system also comprises a decompression unit 7′ with a second data processing system 41 (see also FIG. 6) that is arranged separately from the first data processing system 40. The second data processing system 41 may be comprised or partially comprised by a stationary part of the CT scanner 11. The second data processing system 41 may also be provided separately from the CT scanner 11 or partially separately from the CT scanner 11. The first data processing system 40 and the second data processing system 41 may be connected together for data transmission via a transmission link 27 (communication link 27), in particular a slip ring of the CT scanner 11.

X-ray projections from a plurality directions may be captured using the CT scanner 11. On the basis of the differing attenuation of the X-rays by different regions of the object to be imaged that is introduced, for example by way of a patient table 4, into the capture region of the CT scanner 11, it is possible to draw conclusions as to the material composition of the captured region by way of computer-aided reconstruction methods. The detector data, which in particular represents the X-ray projections, is generated by the CT scanner 11 and transferred via the transmission link 27 from first data processing system 40 to the second data processing system 41.

The first data processing system 40 and the second data processing system 41 are suitable for jointly carrying out a computer-implemented method for compression and decompression of data from the CT device 10 according to one or more example embodiments of the invention. The first data processing system 40 is in this respect set up to carry out a computer-implemented method for compressing data from the CT device 10 according to one or more example embodiments of the invention. In particular, the first data processing system 40 may comprise one or more ASICs and/or one or more FPGAs that are set up to carry out the computer-implemented method according to one or more example embodiments of the invention for compressing data from the computed tomography device 10. The first data processing system 40 may alternatively or additionally comprise one or more CPUs that are suitable for carrying out the computer-implemented method according to one or more example embodiments of the invention for compressing data from the computed tomography device 10.

FIG. 6 shows a flow chart of a computer-implemented method for producing a reconstructed CT image B, wherein a computer-implemented method such as that described in FIG. 5 is carried out by the first data processing system 40 and the fully compressed data 5 is transferred from the first data processing system 40 via the transmission link 27 to the second data processing system 41. Both the second detector data 3b and the fully compressed data 5 comprise in particular integral data, for example multi-dimensional integral data. The precompressed data 4 is restored by the second data processing system 41 on the basis of the fully compressed data 5 by reversal of the lossless compression method and decompressed data 9 is generated by the second data processing system 41 on the basis of the restored precompressed data 4′ by reversal of the lossy compression method. The reconstructed CT image B is generated by the second data processing system 41 as a function of the decompressed data.

For example, the second data processing system 41 may also store the fully compressed data 5 on a temporary storage means 8, in particular for short-term storage. In some embodiments, the second data processing system 41 may generate preprocessed data 9* on the basis of the decompressed data 9 and create the reconstructed CT image B on the basis the preprocessed data 9*. The preprocessed data 9* may be float data, in particular multi-dimensional float data.

Optionally, the second data processing system 41 may store the preprocessed data 9* in a further storage means 8′, for example for long-term storage. Alternatively or in addition, the second data processing system 41 may generate sampled data A for example by performing linear sampling of the preprocessed data 9*, and store the sampled data A in the further memory device 8′, for example for long-term storage. The sampled data A is integral data, in particular multi-dimensional integral data.

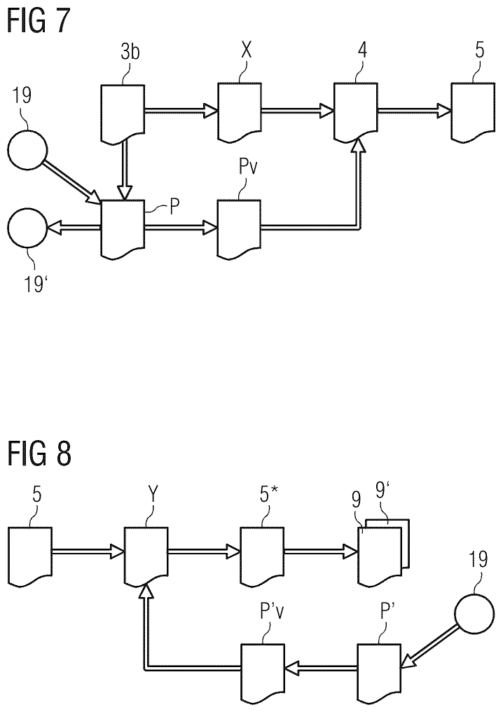

FIG. 7 shows a flow chart of a further computer-implemented method for compressing data from the CT device 10 according to one or more example embodiments of the invention, which may be used, for example, in the computer-implemented method of FIG. 6.

The detector data 3 here in each case comprises a pixel value x for each of a plurality of detector pixels of the X-ray detector and the lossy compression method comprises pixel-by-pixel lossy compression, wherein a respective precompressed V pixel value L(x) is calculated for each detector pixel c of the plurality of detector pixels using a predefined compression function L. FIG. 7 shows the second detector data 3b to be compressed of a current image.

For each detector pixel c, for example, historical V pixel values hc of each surrounding pixel within predefined historical surroundings Hc of the detector pixel c can be calculated from the plurality of detector pixels. A predictor value Pc for the V pixel value for the detector pixel c is calculated as a function of the historical V pixel values hc of the surrounding pixels of the detector pixel c and independently of the V pixel value x itself. The precompressed data 4 is generated as a function of the (precompressed) V pixel value L(x) and the predictor value Pc.

It should be noted that, in practice, the predictor value cannot generally be calculated directly from the V pixel values compressed on a lossy basis. Lossy compression often causes the correlation between the values of two neighboring pixels compressed on a lossy basis to be lost. It is therefore preferred, if the predictor value is a weighted sum, firstly to form the weighted sum of the original pixel values (i.e., the uncompressed pixel values) and then to perform lossy compression of this sum. It should additionally be mentioned in this respect that the “original pixels” may also already be compressed on a lossy basis in a manner of speaking, for example by undersampling the range of values in linear or nonlinear manner. This could be the case, for example, if the detector has 20-bit resolution and 16-bit pixel values are captured due to noise considerations. In this specific example, the detector for example captures data with a bit depth of 20 bits, and these values are undersampled by way of a partially linear/root function, in order to arrive, for example, at a bit depth of 16 bits. After this “compression”, the neighboring pixels are still well correlated. Actual lossy compression takes place by calculating the difference between the current 16-bit value and the 16-bit value of the same pixel in the previous detector readout procedure (frame) and undersampling this difference by a nonlinear function. In this way, 9-bit values are obtained, for example. However, due to this difference calculation, the 9-bit values of two neighboring pixels are as a rule not or only poorly correlated (in particular if multiple readout procedures have taken place, in each case with calculation of the difference from the previous one). Lossy compression may then, for example, be understood either as 20- to 9-bit compression or as 16- to 9-bit compression. The predictor for lossless compression may be calculated on the basis of the 20-bit data or of the 16-bit data, since in these cases the neighboring pixels correlate, but not on the basis of the 9-bit data, since here the correlation between the neighboring pixels is destroyed due to the difference being calculated with the previous readout procedure. In this example, therefore, a predictor is firstly calculated on the basis of the 20-bit data or 16-bit data, and this is then recalculated by way of lossy compression into a 9-bit predictor that is then used for the lossless compression.

The historical surroundings Hc for a detector pixel c are in particular a set of N uncompressed pixel values xi of detector pixels that have already been compressed at the point in time when the detector pixel c is to be compressed. This ensures that these values are available during decompression, when the detector pixel c is to be decompressed. In general in this case it may be a question of pixels that are located in the vicinity of the current pixel c and have a strong correlation with the current pixel value x, but this does not necessarily have to be the case.

The compression function L maps the pixel value x onto a precompressed V pixel value L(x) that is an integer in the interval [0, 2m−1], wherein m is a predefined bit depth. The compression function L in particular fulfills the condition L (L−1(L(x)))=L(x) for each pixel value x. In other words, the inverse compression function L−1 is bijective over the range of integers within [0, 2m−1]. The compression function L may, for example, be a square root function or partly a square root function and partly a linear function. In some implementations, it is also possible for the compression function L to map an argument containing the pixel value x and the historical pixel values hc onto the precompressed V pixel value L(x).

The predictor value Pc may be regarded as an estimate of the pixel currently to be compressed. The predictor value Pc may be a weighted sum or for example be calculated using a trained MLM, such as a trained ANN, in particular a deep ANN. The predictor value Pc is calculated in particular as a function of a set of predefined parameters 19, wherein these may be the respective coefficients of the weighted sum or comprise the respective network parameters, in particular weightings and distortions, of the ANN.

In some implementations, the parameter 19 may be updated, resulting in updated parameters 19′ according to a predefined update rule. Updating may be carried out for each pixel or each predefined group of pixels etc.

If the parameters 19 are coefficients for the weighted sum, updating may proceed for example by minimizing a cost function, which is dependent, for example, on the squared error between the pixel value x and the corresponding predictor value which is summed over a plurality of pixels. If the parameters 19 are network parameters of the ANN, updating may be carried out by further training of the ANN as a function of the predictor value using the pixel value x as ground truth.

Pixel-by-pixel application of the compression function L to the detector data results in precompressed V pixel values L(x), and pixel-by-pixel calculation of the predictor values results in predictors P. Application of the compression function L to the predictors P generates precompressed predictors Pv. The precompressed data 4 can then be generated by calculating a respective difference value between each precompressed V pixel value L(x) and the corresponding precompressed predictor value L(Pc) for the respective detector pixel c.

(L(x)−L(P_{c})+2^{m})mod 2^{m}.

This leads in particular to many different values close to zero or close to 2m−1. The precompressed data 4 thus has low entropy and can therefore be particularly well compressed using entropy coding methods. The fully compressed data 5 is then calculated by application of the lossless compression method, for example ANS, to the precompressed data 4. This may take place frame by frame, or by stack for one stack of frames after the other. The stack of frames may comprise dozens or hundreds of frames, for example 64 frames, 408 frames or 256 frames. It is also possible for just one sub-image to be compressed or for stacks of sub-images to be compressed, for example on the assumption that the external detector regions behave differently from the internal ones.

FIG. 8 shows a flow chart of the decompression part of a computer-implemented method for compression and decompression of data from the CT device 10 according to one or more example embodiments of the invention, which may be used, for example, in the computer-implemented method of FIG. 6. The fully compressed data 5 may be generated in particular as described in relation to FIG. 7.

The decompression process may largely be regarded as a reversal of the compression process. Reversed lossless compression, in particular reversed ANS, is used to generate the restored precompressed data 4′. The lossy compression method is reversed by pixel-by-pixel lossy decompression, wherein a corresponding decompressed pixel value is calculated for each detector pixel of the plurality of detector pixels using L−1. The predictor values are recalculated for each detector pixel as a function of the historical pixel values of the surrounding pixels of the respective detector pixel. The respective decompressed pixel values are calculated as a function of the restored precompressed data 4′ and the recalculated predictor values P′. In particular, the precompressed, recalculated predictor values P′v are precompressed using the compression function L.

In a processing step Y in particular, the respective cumulative value mod 2m is calculated from the recovered difference value for the respective detector pixel and the precompressed, recalculated predictor value P′v. As a result of this process being carried out for all detector pixels and the current image, processed data 5* is generated. The decompressed data 9 is then calculated by application of the inverse compression function L−1 to the cumulative values of the prepared data 5*. FIG. 8 also shows further decompressed data 9′ of the preceding image. It should be noted that the reversed lossy compression is carried out for example image by image and pixel by pixel in the same order in which the lossy compression was carried out. In this way, the predictor values can be repeatedly recalculated, as on lossy compression.

An improved compression ratio is achieved in multiple embodiments of one or more example embodiments of the invention. A prototype for the compression of raw data of a photon-counting CT scanner successfully increased the compression ratio by around 60% compared with existing lossy methods, without the data suffering any further degradation. In particular, data loss was not increased by the lossy part of the method compared with the existing lossy method or, in other words, the increased compression rate is not achieved at the cost of increased data degradation.

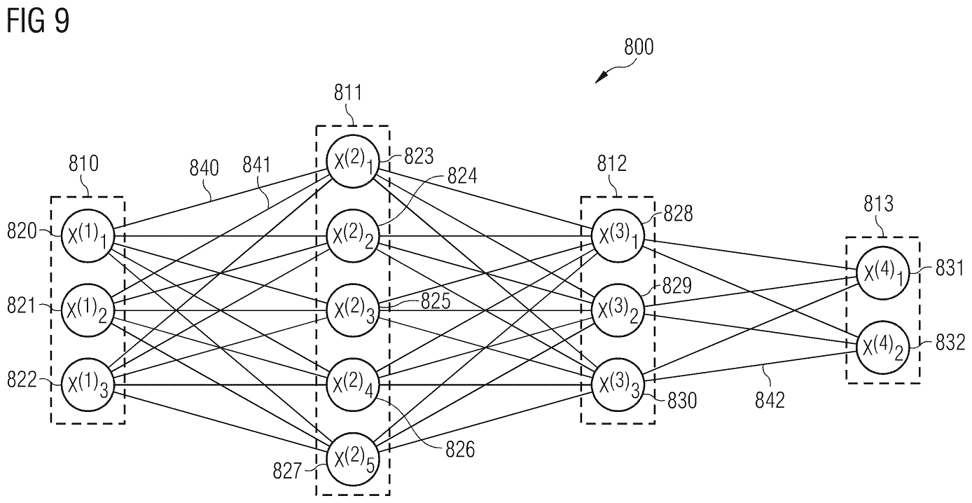

FIG. 9 shows an embodiment of an ANN 800, configured for example as an MLP. The ANN 800 comprises nodes 820, . . . , 832 and edges 840, . . . , 842, wherein each edge 840, . . . , 842 is a directional connection from a first node 820, . . . , 832 to a second node 820, . . . , 832. In general, the first node 820, . . . , 832 and the second node 820, . . . , 832 are different nodes 820, . . . , 832, but it is also possible for the first node 820, . . . , 832 and second node 820, . . . , 832 to be identical. In FIG. 9, the edge 840 is for example a directional connection from node 820 to node 823, and the edge 842 is a directional connection from node 830 to node 832. An edge 840, . . . , 842 from a first node 820, . . . , 832 to a second node 820, . . . 832 is also designated an “incoming edge” for the second node 820, . . . , 832 and an “outgoing edge” for the first node 820, . . . , 832.

In this example, nodes 820, . . . , 832 of the artificial neural network 800 can be arranged in layers 810, . . . , 813, wherein the layers may have an intrinsic order that is introduced by edges 840, . . . , 842 between nodes 820, . . . , 832. In particular, edges 840, . . . , 842 can only be present between adjacent layers of nodes. In the example shown, there is an input layer 810 that consists only of nodes 820, . . . , 822 without incoming edges, an output layer 813 that consists only of nodes 831, 832 without outgoing edges, and hidden layers 811, 812 between input layer 810 and output layer 813. In general, the number of hidden layers 811, 812 can be selected at will. In an MLP, this number is at least one. The number of nodes 820, . . . , 822 within input layer 810 generally relates to the number of input values of the artificial neural network 800, and the number of nodes 831, 832 within output layer 813 normally relates to the number of output values of the artificial neural network 800.

In particular, each node 820, . . . , 832 of artificial neural network 800 can be assigned a real number as its value. In this case, x(n)i designates the value of the ith node 820, . . . , 832 of the nth layer 810, . . . , 813. The values of nodes 820, . . . , 822 of input layer 810 correspond to the input values of artificial neural network 800, while the values of nodes 831, 832 of output layer 813 correspond to the output value of artificial neural network 800. Each edge 840, . . . , 842 may furthermore comprise a weight that is a real number, in particular the weight is a real number within the range [−1, 1] or within the range [0, 1]. In this case, w(m,n)i,j designates the weight of the edge between the ith node 820, . . . , 832 of the mth layer 810, . . . , 813 and the jth node 820, . . . , 832 of the nth layer 810, . . . , 813. The abbreviation w(n)i,j is moreover defined for the weight w(n,n+1)i,j. The output values of the neural network 800 are calculated in particular by propagating the input values through the neural network 800. In particular, the values of nodes 820, . . . , 832 of the (n+1)th layer 810, . . . , 813 are calculated on the basis of the values of nodes 820, . . . , 832 of the nth layer 810, . . . , 813 by

$${x}_{j}^{( n + 1 )} = f ( {\sum }_{i} {x}_{i}^{( n )} {w}_{i , j}^{( n )} ) .$$

The function f here is denoted transfer function or activation function. Known transfer functions are the step function, the sigmoid function, for example, the logistic function, the generalized logistic function, the hyperbolic tangent, the arctangent function, the error function, the smoothstep function, or rectifier functions. The transfer function is used for normalization, for example. The values are in particular propagated layer-by-layer through the neural network 800, wherein the values of input layer 810 are provided by the input of neural network 800, wherein the values of the first hidden layer 811 can be calculated on the basis of the values of input layer 810 of the neural network 800, wherein the values of the second hidden layer 812 can be calculated on the basis of the values of the first hidden layer 811, etc.

The neural network 800 has to be trained with training data in order to define the values w(m,n)i,j for the edges. The training data in particular comprises training input data and training output data (designated ti). In a training step, the neural network 800 is applied to the training input data in order to generate calculated output data. In particular, the training data and the calculated output data comprise a number of values, wherein this number corresponds to the number of nodes of the output layer. In particular, a comparison between the calculated output data and the training data is used to recursively adapt the weights within the neural network 800 (backpropagation algorithm). In particular, the weights are modified according to

$${w}_{i , j}^{′ ( n )} = {w}_{i , j}^{( n )} - γ {δ}_{j}^{( n )} {x}_{i}^{( n )} ,$$

wherein γ is a predefined learning rate, and the numbers δ(n)j can be recursively calculated as

$${δ}_{j}^{( n )} = ( {\sum }_{k} {δ}_{k}^{( n + 1 )} {w}_{j , k}^{( n + 1 )} ) {f}^{′} ( {x}_{i}^{( n )} {w}_{i , j}^{( n )} )$$

on the basis of δ(n+1)j, if the (n+1)th layer is not the output layer 813, and

$${δ}_{j}^{( n )} = ( {x}_{j}^{( n + 1 )} - {t}_{j}^{( n + 1 )} ) {f}^{′} ( {x}_{i}^{( n )} {w}_{i , j}^{( n )} ) ,$$

if the (n+1)th layer is the output layer 813, wherein f′ is the first derivative of the activation function and t(n+1)j is the comparison training value for the jth node of output layer 813.

It should finally once again be noted that the invention described in detail above merely involves exemplary embodiments which can be modified in the most varied manner by a person skilled in the art without departing from the scope of the invention. Furthermore, use of the indefinite article “a” does not rule out the possibility of a plurality of the features in question also being present. Likewise, terms such as “unit” do not rule out the possibility of the components in question consisting of a plurality of interacting subcomponents which may optionally also be spatially distributed. The term “a number” should be taken to mean “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’, the term ‘data processing device’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device processor, Central Processing (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as an data processing system) or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, of additions and substitutions example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

### LIST OF REFERENCE SIGNS

1 Raw data
3 Detector data
3a First detector data
3b Second detector data
4 Precompressed data
4′ Restored precompressed data
5 Fully compressed data
5* Processed data
6 Computing means (also referred to as a second processing device)
7 Compression unit (also referred to as a compression device)
7′ Decompression unit (also referred to as a decompression device)
8 Temporary storage means
8′ Further storage means
9 Decompressed data
9′ Decompressed data
9* Preprocessed data
10 Computed tomography device
11 Gantry/CT scanner
12 Patient
13 Patient couch
14 Patient table
15 X-ray emitter
16 x-ray detector
17 Control means
18 Display means
19 Rotating portion
20 Processing means (also referred to as a first processing device)
21 Processing unit
22 Transformation unit
23 Spatial resolution reduction unit
24 Selection unit (also referred to as a selector)
25 Transfer unit (also referred to as a transfer device)
26 Communication path
27 Communication link/transmission link
28 Stationary portion
29 Temporary storage unit
31 Buffer
32 Preparation unit
33 Reconstruction unit
40 Data processing system
41 Data processing system
800 Neural network/ANN
810 Input layer
811 Hidden layer
812 Hidden layer
813 Output layer
820, . . . , 832 Nodes
840, . . . , 842 Edges
A Sampled data
B CT image
P Predictor
Pv Precompressed predictor
P′ Recalculated predictor
P′v Precompressed recalculated predictor
S1-S10 Method steps
X Pixel value
Y Processing step

1. A method for operating a computed tomography device that has a stationary portion, a rotatable portion that is rotatable relative to the stationary portion, and a communication path including a wireless communication link between the stationary portion and the rotatable portion, wherein the rotatable portion has an X-ray detector configured to acquire raw data and a first processing device configured to determine detector data based on the raw data, wherein the stationary portion has a second processing device configured to process the detector data, the method comprising:
during an imaging procedure, dividing the detector data by way of a selection unit of the first processing device into first detector data and second detector data;
transferring the first detector data in a first data stream via the communication path from the first processing device to the second processing device and, after conclusion of a raw data acquisition by the X-ray detector, the first detector data being usable for reconstruction of an evaluatable CT image data set; and
temporarily storing the second detector data in a temporary storage and, after conclusion of the raw data acquisition by the X-ray detector and the transferring the first detector data, transferring the second detector data in a compressed form in a second data stream via the communication path from the first processing device to the second processing device, wherein for compressed transfer of the second detector data,
precompressed data is generated by compression of the second detector data using a lossy compression method, and fully compressed data is generated by application of a lossless compression method to the precompressed data.

2. The method of claim 1, wherein
the detector data comprises a respective pixel value for each detector pixel of a plurality of detector pixels of the X-ray detector and the lossy compression method comprises pixel-by-pixel lossy compression,
a respective precompressed pixel value of the precompressed data is calculated for each detector pixel of the plurality of detector pixels.

3. The method of claim 2, wherein the lossless compression for each detector pixel of the plurality of detector pixels comprises:
receiving historical precompressed pixel values for each surrounding pixel within predefined historical surroundings of the respective precompressed pixel value of the detector pixel;
calculating a predictor value for the respective precompressed pixel value as a function of the historical precompressed pixel values of the surrounding pixels of the respective precompressed pixel value of the detector pixel and independently of the precompressed pixel value of the respective detector pixel; and
generating the fully compressed data as a function of the precompressed pixel values for the respective detector pixel and of the predictor value for the respective precompressed pixel value.

4. The method of claim 3, wherein, for each detector pixel of the plurality of detector pixels,
a precompressed predictor value is calculated based on the predictor value for the precompressed pixel value of the respective detector pixel;
a difference value between the precompressed pixel value for the respective detector pixel and the predictor value for the respective detector pixel is calculated; and
the fully compressed data is generated as a function of the difference values for the plurality of detector pixels.

5. The method of claim 4, wherein, after transfer of the compressed second detector data,
the precompressed data is restored based on the fully compressed data by reversal of the lossless compression method and decompressed data is generated based on the restored precompressed data by reversal of the lossy compression method.

6. The method of claim 5, wherein
a further precompressed predictor value is calculated based on the predictor value; and
for each precompressed pixel value for each detector pixel of the plurality of detector pixels, the difference value for the precompressed pixel value is recovered based on fully compressed data, a cumulative value of the difference value for the precompressed pixel value and the further precompressed predictor value for the respective detector pixel is calculated, and the respective precompressed pixel value is calculated as a function of the cumulative value.

7. The method of claim 1, wherein the second detector data is compressed before the second detector data is temporarily stored.

8. The method of claim 1, wherein information preparing a next imaging procedure is transmitted to the rotatable portion during transfer of the compressed second detector data.

9. The method of claim 1, wherein, prior to being compressed, the second detector data is divided into sub-data sets and the precompressed data is correspondingly generated from the sub-data sets by separate compression of the sub-data sets using the lossy compression method and fully compressed data is generated from the precompressed data using a lossless compression method.

10. A system for operating a computed tomography device that has a stationary portion, a rotatable portion that is rotatable relative to the stationary portion, and a communication path including a wireless communication link between the stationary portion and the rotatable portion, wherein the rotatable portion has an X-ray detector configured to acquire raw data and a first processing device configured to determine detector data based on the raw data, wherein the stationary portion has a second processing device configured to process the detector data, the system comprising:
a selection unit configured to divide the detector data during an imaging procedure into first detector data and second detector data, after conclusion of raw data acquisition by the X-ray detector, the first detector data being usable for reconstruction of an evaluatable CT image data set;
a temporary storage configured to temporarily store the second detector data;
a transfer unit configured to transfer the first detector data in a first data stream via the communication path from the first processing device to the second processing device and for compressed transfer of the second detector data in a second data stream via the communication path from the first processing device to the second processing device after conclusion of raw data acquisition by the X-ray detector; and
a compression unit configured to compress the second detector data such that, for compressed transfer of the second detector data, precompressed data is generated by compression of the second detector data using a lossy compression method, and fully compressed data is generated by application of a lossless compression method to the precompressed data.

11. The system of claim 10, further comprising:
a decompression unit configured to restore the precompressed data based on the fully compressed data by reversal of the lossless compression method and to generate decompressed data based on the restored precompressed data by reversal of the lossy compression method.

12. A controller for a computed tomography device, comprising:
the system of claim 10.

13. A computed tomography device, comprising:
the controller of claim 12.

14. A non-transitory computer program product comprising instructions, when executed by a computer, cause the computer to perform the method of claim 1.

15. A non-transitory computer-readable storage medium comprising instruction, when executed by a computer, cause the computer to perform the method of claim 1.

16. The method of claim 1, wherein the transferring the first detector data transfer the detector data in the form of a direct real-time transfer.

17. The method of claim 2, wherein the pixel-by-pixel lossy compression for each detector pixel of the plurality of detector pixels comprises calculation of the respective precompressed pixel value using a predefined compression function, an argument of the predefined compression function is dependent on the respective pixel value.

18. The method of claim 3, wherein the receiving, the calculating, and the generating are at least in part performed during the transferring the first data stream.

19. The method of claim 4, wherein the predictor value for the precompressed pixel value of the respective detector pixel
is calculated as a weighted sum of the historical precompressed pixel values; or
is obtained by application of a trained machine learning model to the historical precompressed pixel values.

20. The method of claim 5, wherein
reversal of the lossless compression method comprises lossless decompression, wherein a respective precompressed pixel value is calculated for each detector pixel of the plurality of detector pixels, and
lossless decompression for each precompressed pixel value for each detector pixel of the plurality of detector pixels comprises receiving the received historical precompressed pixel values of each surrounding pixel within the historical surroundings of the respective detector pixel, recalculating the predictor value for the respective detector pixel as a function of the recovered historical precompressed pixel values of the surrounding pixels of the respective detector pixel, and calculating the respective precompressed pixel value as a function of the fully compressed data and the recalculated predictor value for the respective detector pixel.
